(19) United States
(12) Patent Application Publication
(10) Pub. No.: US 2022/0298450 A1
(43) Pub. Date: Sep. 22, 2022

(54) METHOD FOR EXTRACTION OF A RAW MATERIAL TO PRODUCE A FORMULATION RICH IN PHYTOCHEMICALS

(71) Applicant: Nutragreen - Technical & Research Solutions, HJÄRUP (SE)

(72) Inventor: Arwa MUSTAFA, Hjärup (SE)

(21) Appl. No.: 17/619,617

(22) PCT Filed: Jun. 16, 2020
(86) PCT No.: PCT/SE2020/050626

§ 371 Date: Dec. 16, 2021

(30) Foreign Application Priority Data
Jun. 20, 2019 (SE) 1950765-6

(51) Int. Cl. C11B 1/10; A61Q 19/00; A61Q 19/08; A61K 8/64; A61K 8/92; A61K 8/63; B01D 11/02; A23D 9/02

(52) U.S. Cl. CPC C11B 1/104; C11B 1/106; A61Q 19/007; A61Q 19/08; A61K 8/645; A61K 8/922; A61K 8/63; B01D 11/0203; B01D 11/0265; A23D 9/02

(57) ABSTRACT

The present invention describes a method for extraction of a raw material or a combination of raw materials to produce a formulation (NEAT™) rich in phytochemicals and which is based on or rich in oil and proteins, said method comprising—bringing the raw material or combination of raw materials into contact with a solvent being carbon dioxide and which optionally is assisted with one or more co-solvents being GRAS (generally recognized as safe), such as but not limited to water, ethanol, dimethyl carbonate; —performing an extraction in high pressure conditions ranging from subcritical to super critical conditions in carbon dioxide in a pressure range of 250-800 bars and in a temperature range of 15-45° C. to provide a formulation being rich in phytochemicals, e.g. lipids, phenolics, terpenoids and alkaloids and proteins wherein a residue obtained in the method for extraction is subjected to extraction in carbon dioxide at supercritical conditions in a pressure above 72 bars and 31° C., and wherein pressurized hot water is used as a co-solvent or after the extraction with carbon dioxide.

### FIELD OF THE INVENTION

The present invention relates to a method for extraction of a raw material to produce different formulations rich in phytochemicals (NEAT™ (Natural Environmentally Adapted Technology)).

### TECHNICAL BACKGROUND

There are several known methods for extraction of raw materials and crude oils to produce refined oils. The present invention is directed to such a method.

To provide some known methods in the prior art the following could be mentioned. For instance, in CN103484238A there is disclosed a method for extracting cibora jadalna oil by using supercritical carbon dioxide. Pulverized cibora jadalna powder is extracted in supercritical carbon dioxide at a pressure of 20-35 MPa and at a temperature of 40-45° C., and wherein the separation pressure is 8-10 MPa and at a temperature of 45-50° C.

There are also other examples where vegetable oil materials are extracted in sub- or supercritical carbon dioxide. Examples of such methods are disclosed in CN102138960A, CN101831351A and CN103450135A.

The present invention is directed to an improved extraction method for certain fruits, vegetables and cereals or a combination thereof to produce phytochemicals formulation that are based on or rich in oils and possibly plant-proteins.

### SUMMARY OF THE INVENTION

The stated purpose above is achieved by a method for extraction of a raw material or a combination of raw materials to produce a formulation rich in phytochemicals, and preferably plant-proteins and free amino acids, which is based on or rich in oil, said method comprising
bringing the raw material or combination of raw materials into contact with a solvent being carbon dioxide and which optionally is assisted with one or more co-solvent(s) being GRAS (generally recognized as safe), such as but not limited to water, ethanol, dimethyl carbonate;
performing an extraction in high pressure conditions ranging from subcritical to super critical conditions in carbon dioxide in a pressure range of 250-800 bars and in a temperature range of 15-45° C. to provide an extract being rich in phytochemicals, e.g. lipids, phenolics, terpenoids and alkaloids wherein a residue obtained in the method for extraction is subjected to extraction in carbon dioxide at supercritical conditions in a pressure above 72 bars and 31° C.,
   and wherein pressurized hot water is used as a co-solvent or after the extraction with carbon dioxide.

The method according to the present invention has several advantages. First of all, at the conditions provided above it is possible to maximize the extraction of oils rich/based phytochemicals. Secondly, no dangerous chemicals are involved, and only environmentally friendly and safe chemicals (GRAS_{—} generally recognized as safe), such as water, ethanol, dimethyl carbonate, and carbon dioxide are involved or any GRAS solvent/s. This and the general steps of the method according to the present invention implies that the method may be regarded as clean and green tech.

Furthermore, the end product obtained is based 100% on natural raw material. This end product is ultra-pure and chemical free. Moreover, the quality of the end product is very high. Furthermore, the method according to the present invention reduces waste and decreases costs when being compared with known methods used today. The end product has positive health functional properties.

Below there is provided some information on certain published documents.

First of all, in JP2016011400A there is disclosed a method for producing a glyceroglycolipid-containing composition in which a supercritical carbon dioxide and a polar solvent may be used together as an extraction solvent. A glyceroglycolipid-containing composition as described has a little content of reducing sugar for the production of a soft capsule formulation. The present invention, however, is directed to the production of a formulation rich in phytochemicals. Already in this regard there is a clear and important difference. Moreover, the method according to the present invention also involves steps, such as also including a subsequent extraction of a residue in carbon dioxide at supercritical conditions in a pressure above 72 bars and 31° C. Furthermore, according to the present invention pressurized hot water or other GRAS solvent, preferably pressurized hot water, is used as a co-solvent or after the extraction with carbon dioxide.

Secondly, in Determination of Optimum Conditions for Supercritical Fluid Extraction of Carotenoids from Carrot (Daucus carota L.) Tissue, J. Agri. Food Chem. 1995, 43, 2876-2878 (Barth et al.) there is disclosed extraction of antioxidant vitamins carotenes. The supercritical extraction part of the study is based on freeze drying of the sample. There are several key differences when comparing the method used in this study with the method according to the present invention. Such non-limiting difference examples are, the starting raw material used, the method employed and steps involved in the method and as such of course also the end result.

Moreover, extraction of oil from almonds, not directed to phytochemicals, are also disclosed in e.g. Development of Pressurized Extraction Processes for Oil Recovery from Wild Almond (Amygdalus scoparia) (Balvardi et al.) and High-power ultrasonic system for the enhancement of mass transfer in supercritical CO2 extraction processes (Riera et al.). In these cases, the same key differences as mentioned above are valid when comparing with the method according to the present invention.

### SPECIFIC EMBODIMENTS OF THE INVENTION

Below some specific embodiments of the present invention are disclosed and discussed further.

According to one specific embodiment of the present invention, one or more green co-solvents are used in the extraction. Furthermore, according to yet another embodiment of the present invention, the one or more green co-solvents is water, ethanol, dimethyl carbonate or a combination thereof.

Moreover, and as stated above, according to the present invention, a residue obtained in the method for extraction is subjected to extraction in carbon dioxide at supercritical conditions in a pressure above 72 bars and 31° C. It should be noted that also a new starting material may be combined with the residue for this additional step.

Co-solvents may be used in the method according to the present invention. This implies that such one or more co-solvents may be used together with carbon dioxide. This may be preferable in some applications. According to the present invention, pressurized hot water or other GRAS solvent is used as a co-solvent or after the extraction with carbon dioxide. Using pressurised hot water or ethanol after the extraction with supercritical carbon dioxide may be performed for better isolation/fractionation possibilities of different fractions. For instance, this may be beneficial for the extraction of hydrophilic molecules.

Furthermore, according to yet another specific embodiment of the present invention, the extraction is carried out under sonication and/or ultrasound assisted extraction.

Moreover, the method according to the present invention suitably also comprises a separation step. According to one specific embodiment of the present invention, the method comprises a separation step which is a membrane separation step. Membrane filtration is suitably used as the in-line separation technology.

Furthermore, also polarity control may be used in the method according to the present invention. Therefore, according to one specific embodiment of the present invention, the method involves polarity control. Some lipid compounds are slightly polar, such as phospholipids and glycolipids, and they can be a challenge to be extracted using near supercritical carbon dioxide in itself. Therefore, and as mentioned, the method according to the present invention may involve the addition of a co-solvent, like ethanol or another type of green solvent. This may be important to match the polarity. The extraction system used is suitably equipped with a pump for co-solvent addition. It may be important to try to add the minimum amount of the solvent by increasing the pressure since the combination of temperature and pressure can enhance the polarity and solubility in supercritical carbon dioxide. The co-solvent may be removed from the oil-based formulation by using a rotary evaporator, hot-water bath or using a stream of nitrogen.

Also the starting material is relevant in relation to the method according to the present invention. According to one specific embodiment of the present invention, the raw material or combination of raw materials are chosen from any of Frankincense, Baobab, Moringa, Oats, Blueberries, Rubus chamaemorus, Raspberry, Grape vine, Carrots, Tomatoes, Marigold, curly kale, olives and olive leaves or a combination of any of these.

Below, there is provided a list of different raw materials which is usable according to the present invention.
1. Frankincense: Family: Burseraceae Scientific name: Boswellia sacra
2. Baobab: Scientific name: Adansonia Family: Malvaceae
3. Moringa: Family: Moringaceae Scientific name: Moringa oleifera
4. Oats: Family: Poaceae Scientific name: Avena sativa′
5. Blueberries: Family: Ericaceae, name: Vaccinium sect. Cyanococcus
6. Rubus chamaemorus Family: Rosaceae; name cloudberry, nordic berry
7. Raspberry Family: Rosaceae, name: Rubus idaeus
8. Grape vine, Family: Vitaceae, name: Vitis vinifera,
9. Carrots (Daucus carota); Family Apiaceae; name: D. c. subsp. Sativus
10. Tomatoes; Family Solanaceae; name S. lycopersicum
11. Curly Kale; Family; Brassica oleracea, name: Kale or leaf cabbage
12. Olives and olive leaves, Family; Oleaceae, name Olea europaea
13. Marigold; Family Asteraceae, name; Tagetes.
14. Buckwheat, Fagopyrum esculentum, Family: Polygonaceae
15. Amaranthus, collectively known as amaranth. Family: Amaranthaceae
16. Quinoa Chenopodium quinoa Family: Amaranthaceae

According to one specific embodiment of the present invention, the method produces an oil-based formulation comprising phytochemicals and/or phytosterols. Furthermore, according to yet another specific embodiment of the present invention, the method produces an oil-based formulation comprising at least any three of the following components:
one or more glycerolipids, phospholipids, glycolipids or terpenoids, or a combination thereof;
omega 3, 6 or 9, or a combination thereof;
one or more antioxidants, e.g. Lutein, Oleuropein, or a range of carotenoids;
at least one plant-protein, e.g. lysine, tryptophan, threonine, aspartic acid, and other possible essential amino acids and sulphur-containing amino acids, and free amino acids;
vitamin A, E and/or vitamin D; and
one or more phenolic compounds.

The end product obtainable by the method according to the present invention has a wide range of health benefiting compounds, such as phytosterols, Omega 3, 6 & 9, antioxidants, vitamin A, E & D and essential fatty acids, anti-oxidants, vitamins, minerals, carotenes including lycopene, other carotenoids; in addition to phytosterols and other important nutrients that play an important role in the health and radiance of the skin. One of the end products according to the present invention has excellent skin penetration and is non-greasy. Moreover, it may deeply nourish, repair dry skin, improve skin elasticity and decrease roughness. Furthermore, it should be noted that also anti-inflammatory and/or anti-microbial active compounds are suitably involved in the end product according to the present invention. It should be noted that the present invention may also involve adding other substances to the end product / formulation. This may e.g. be intended to enrich the formulation with plant-based proteins and dietary fibers. Some examples are Buckwheat, Fagopyrum esculentum, Family: Polygonaceae Amaranthus, collectively known as amaranth, Family: Amaranthaceae Quinoa Chenopodium quinoa, Family: Amaranthaceae.

It should be noted that an end product according to the present invention may be used in different ways and in different applications. Suitable areas are in skin-care products and cosmetic products, as should be understood from above, but also food applications, functional foods, nutraceuticals and pharma applications are possible.

It should be noted that an end product according to the present invention may be used in other health promoting functions e.g. lutein for eye health and oleuropein for neurodegenerative diseases.

Some interesting alternatives of raw material components are discussed further below in relation to the examples.

### DETAILED DESCRIPTION OF THE DRAWINGS AND EXAMPLES

Extractions was performed in accordance with the method according to the present invention. A lab-scale supercritical fluid extraction system (Isco™) was used in a pressure range 300-800 bars, a temperature range 35-45° C. and a time of 15-30 minutes.

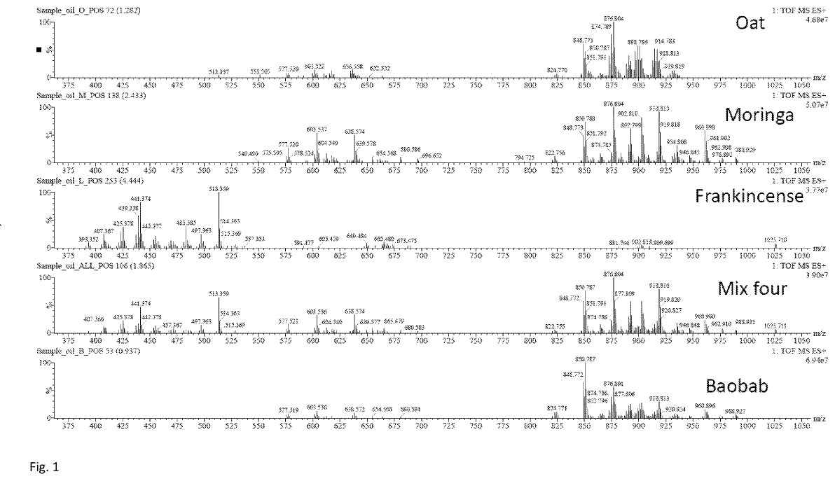

In FIG. 1 there is shown chromatograms of extracted oils obtained in accordance with the present invention. The following is valid here: 1) Where it states Sample_oil_O then the chromatogram illustrates the content from Oats. 2) Where it states Sample_oil_M then the chromatogram illustrates the content from Moringa. 3) Where it states Sample_oil_L then the chromatogram illustrates the content from Frankincense. 4) Where it states Sample_oil_B then the chromatogram illustrates the content from Baobab. 5) Where it states Sample_oil_ALL then the chromatogram illustrates the content from a combination of raw materials from Moringa, Oats, Frankincense and Baobab. This is case where these raw materials were mixed and extracted at the same time.

As may be seen from FIG. 1 for the oils extracted in accordance with the present method Moringa has higher amount of DAGs and steroid esters. The resulted mixture had a wide range of compounds including glycerolipids, phospholipids and glycolipids and terpenoids. By mixing Moringa and Baobab, there was obtained a homogeneous oil-based formulation with a unique combination of Boswellic acids, diglycerides, triglycerides, phospholipids and steroid esters.

In relation to these suitable raw materials to be used when performing the method according to the present invention the following may be mentioned. Moringa (M) oil is rich in a wide range of TAGs (triglyceride) &phospholipids and contains as well DAGs (diglyceride), and steroid esters. Baobab oil is rich in a wide range of TAGs (triglyceride) and phospholipids. Frankincense is rich in a wide range of Boswellic acids. This implies that a resulted mixture of oils from Oats, Moringa, Baobab and Frankincense has a wide range of functional compounds including glycolipids, phospholipids and glycolipids and terpenoids.

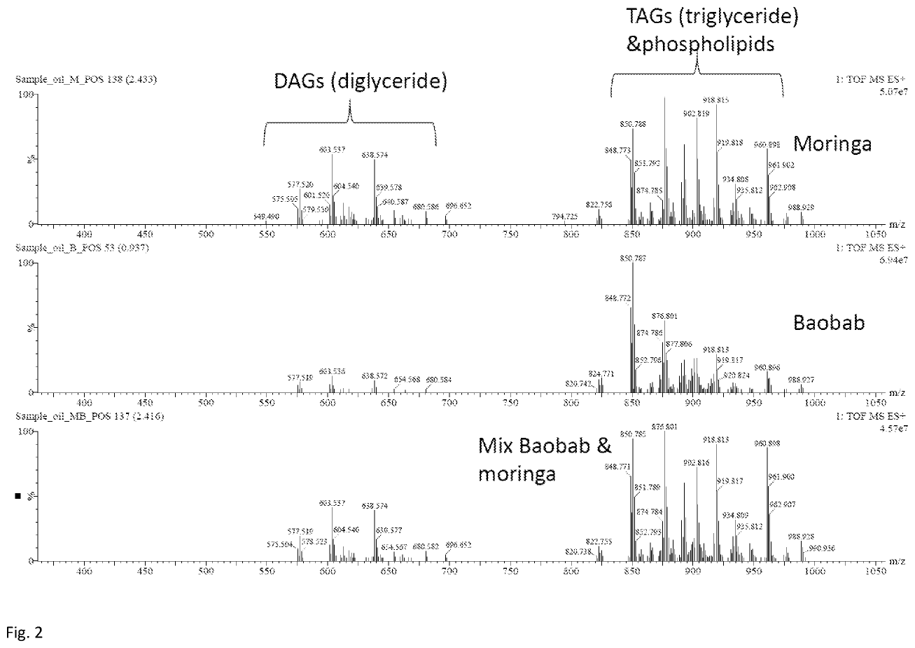

In FIG. 2 there is shown the chromatogram resulting from similar tests as according to FIG. 1, but in this case Moringa and Baobab was mixed. By mixing Moringa and Baobab, there was obtained a homogeneous oil-based formulation with a unique combination of diglycerides, triglycerides, phospholipids and steroid esters.

In relation to suitable raw materials to be used, some mentioned above and others not, the following may also be mentioned. In relation to olives, olives contain concentrations of up to 140 mg per g on a dry matter basis in olives and 60-90 mg per g of dry matter in the leaves of Oleuropein, which is a phenolic compound in olive cultivars and can reach. This may be of interest in relation to the present invention. Moreover, CURLY KALE, which is also usable according to the present invention, contains 20 mg per 100 g raw material Lutein & Zeaxanthin. Furthermore, Frankincense rich has oil content of about 30% mainly rich in Boswellic acids and exhibits a range of microbial activities. Moreover, bilberries have an oil content in the order of 15-20% mainly rich in about 6-9 types of free fatty acids (FFA), e.g. linoleic acid; tocopherols; phytosterols; squalene; unsaturated oils and phenolics. Also this is of interest to use when performing the method according to the present invention for suitable applications. Moreover, Baobab Seed Oil, also of interest according to the present invention, has a content is in the order of 20% mostly rich in Palmatic acid, Oleic acid. This is also reported to have a high content of essential fatty acids e.g. vitamins A, D, E and F.

1. A method for extraction of a raw material or a combination of raw materials to produce a formulation rich in phytochemicals, and preferably plant-proteins, and which is based on or rich in oil, said method comprising
bringing the raw material or combination of raw materials into contact with a solvent being carbon dioxide and which optionally is assisted with one or more co-solvent(s) being GRAS (generally recognized as safe), such as but not limited to water, ethanol, dimethyl carbonate;
performing an extraction in high pressure conditions ranging from subcritical to super critical conditions in carbon dioxide in a pressure range of 250-800 bars and in a temperature range of 15-45° C. to provide a formulation being rich in phytochemicals, e.g. lipids, phenolics, terpenoids and alkaloids,
wherein a residue obtained in the method for extraction is subjected to extraction in carbon dioxide at supercritical conditions in a pressure above 72 bars and 31° C., and wherein pressurized hot water is used as a co-solvent or after the extraction with carbon dioxide.

2. The method according to claim 1, wherein one or more green co-solvents are used in the extraction.

3. The method according to claim 1, wherein the one or more green co-solvents is water, ethanol, dimethyl carbonate or a combination thereof.

4. The method according to claim 1, wherein the extraction is carried out under sonication and/or ultrasound assisted extraction.

5. The method according to claim 1, wherein the method comprises a separation step which is a membrane separation step.

6. The method according to claim 1, wherein the method involves polarity control.

7. The method according to claim 1, wherein the raw material or combination of raw materials are chosen from any of Frankincense, Baobab, Moringa, Oats, Blueberries, Rubus chamaemorus, Raspberry, Grape vine, Carrots, Tomatoes, Marigold, curly kale, olives and olive leaves or a combination of any of these.

8. The method according to claim 1, wherein the method produces an oil-based formulation comprising phytochemicals and/or phytosterols.

9. The method according to claim 1, wherein the method produces an oil-based formulation extract comprising at least any three of the following components:
one or more glycerolipids, phospholipids, glycolipids or terpenoids, or a combination thereof;
omega 3, 6 or 9, or a combination thereof;
one or more antioxidants;
vitamin A, E and/or vitamin D;
at least on plant-protein and free amino acids; and
one or more phenolic compounds.
